(19) United States
(12) Patent Application Publication
(10) Pub. No.: US 2006/0275505 A1
(43) Pub. Date: Dec. 7, 2006

(54) Method and composition for increasing the alkalinity of the body

(76) Inventor: Ignacio Cisneros, Odessa, TX (US)

Correspondence Address: LAUBSCHER & LAUBSCHER, P.C., 1160 SPA ROAD, SUITE 2B, ANNAPOLIS, MD, 21403, US

(21) Appl. No.: 11/145,726

(22) Filed: Jun. 6, 2005

(51) Int. Cl. A61K 33/32; A61K 33/24; A61K 33/06; A61K 33/00

(52) U.S. Cl. 424/641; 424/650; 424/684; 424/722

(57) ABSTRACT

A composition for increasing alkalinity in the body containing water, a source of alkalinity, and silicon.

### FIELD OF THE INVENTION

The present invention relates to a method and composition for increasing the body's alkalinity. More particularly, the method entails administration of a safe, easy to use, and inexpensive beneficial alkaline composition containing silicon to ameliorate a wide variety of ailments, diseases, and conditions.

### BACKGROUND OF THE INVENTION

There is a specific growing awareness that the whole body is one organism, intertwined, and must be treated as a whole entity, rather than just as various unrelated parts. The alternative and mainstream health communities have demonstrated a growing awareness of the need for humans and other animals to maintain optimal pH levels in the body. The higher the pH reading, the more alkaline and oxygen-rich a bodily fluid becomes. Optimal fluid pH levels, ranging from 7 to 7.4, are essential in maintaining a healthy cellular environment. Such levels are crucial for maximizing the absorption of vitamins and minerals, ensuring proper and sufficient elimination of waste materials, and sustaining conditions generally hostile to disease formation or progression.

More specifically, pH is the negative logarithm of the hydrogen ion concentration. (This statement is not quite accurate, as pH depends on hydrogen ion activity rather than concentration). Thus, gastric fluid that contains 0.1 M hydrochloric acid has a pH of 12.0, hydrochloric acid being a strong acid that is almost completely dissociated into hydrogen ions and chloride ions. The pH of lysosomes is approximately 5.0, and the pH of the blood is approximately 7.4.

A measurement of pH is a measurement of the number of negative hydroxyl (OH⁻) ions compared to the number of positive hydrogen ions (H⁺) in the human body. The higher the pH reading, the more alkaline and oxygen rich the bodily fluid. Maintaining the recommended slightly alkaline pH of 7 to 7.4 is elemental for optimal survival.

When the pH of the body drops, i.e., becomes more acid; the body's ability to absorb vitamins, minerals and other nutrients is compromised. Acidic pH in the body adversely affects energy production in the cells, such as the cell's ability to repair itself and its ability to detoxify.

Until the discovery of the instant invention, there remained a dearth of inexpensive, safe, easy to use high alkaline products available to consumers. Most products sold as alkaline water require consumers to process tap water through expensive, multi-stage ionizing filers, membranes, carbon filters, electrodes, etc. One patented product, advertised on the internet as alkaline water, contains potassium hydroxide and sodium hydroxide with distilled water. The product is designed to be used at pH levels between 10 and 10.5. However, the potassium hydroxide is toxic if inhaled or ingested, and sodium hydroxide is corrosive to human and animal tissue. Prices for this product ranged from $15-$25 for a 1.2 oz bottle.

Similarly, many alternative cancer treatments purport to rely on the health benefits of alkalinity. Others promote hygroscopic, oxygen-carrying benefits. Such treatments include DMSO, cesium, rubidium, vegetable and fruit juicing regimens, and vitamins. DMSO, a by-product of the wood industry, has shown promise in treating internal cystitis, but the FDA has repeatedly refused further studies of DMSO following an isolated report of a deadly allergic reaction to the compound. Cesium and rubidium, both mined minerals, react explosively with air and water. Fruit and vegetable juicing regimens require the patient/consumer to devote a significant amount of time and money to procuring and preparing the necessary juicing ingredients. Vitamin supplementation, while arguably effective, is expensive, and may result in overdose if not carefully monitored.

### SUMMARY OF THE INVENTION

It is an object of the present invention to overcome the aforementioned deficiencies in the prior art.

It is another object of the present invention is to provide a safe, effective, and inexpensive composition of matter that assists the human (or other animal body) in maintaining bodily fluids at pH levels optimal for survival, i.e., between 7 and 7.4.

Another object of the present invention to provide an alkaline silicon solution of r promoting health.

A further object of the invention is to provide simple and inexpensive means for producing and administering said compositions to a wide variety of individuals in a wide variety of environments, including clinical and home settings.

A still further object of the present invention is to provide means for determining optimal amounts of the inventive composition to treat specific ailments or conditions in specific individuals.

Yet another object of the present invention is to provide and alkaline, silicon-containing solution for promoting health.

The composition of the present invention contains:
a. 1 to 8 parts by molecular weight of silicon, with optional aluminum, tin, or zinc
b. 8 to 15 parts by molecular weight of R.O. water
c. 1-5 parts by molecular weight of at least one alkaline hydroxide, such as calcium hydroxide, lithium hydroxide, magnesium hydroxide, potassium hydroxide, or sodium hydroxide or a sodium silicate salt, which supplies both silicon and alkalinity.

The silicon, aluminum, tin, and zinc can be provided in the form of physiologically acceptable salts, such as aluminum hydroxide, stannic oxide, zinc sulfate, and sodium silicate.

The pH of the composition is approximately 13.5, although the composition is usually diluted to about 0.1 to about 5.0% by weight with water.

While other ions can be present in the solution of the present invention, the most important ions are sodium and silicon. A 1% silicon solution has 0.057 mg/ml silicon and 0.04 mg/ml solution in water. A 2% silica solution contains 0.110 mg/ml silicon and 0.08 mg/ml sodium, remainder water. In general, the silicon solution can be used in concentrations from about 0.1 to about 5% silicon.

The silicon solution of the present invention is non-toxic, non-corrosive, non-hazardous, non-combustible, and environmentally friendly. The silicon solution of the present invention can reduce and/or eliminate putrid odors of decomposition in bodies and other organic matter associated with embalming, all types of funeral home services, sewage treatment plants, swine yards, cattle yards, and others.

The silicon solution of the present invention is capable of aiding the body's ability to absorb vitamins and minerals not only to reduce and/or eliminate illnesses, but also to replace in the body vitamins and minerals required to build the body's immune system. The silicon solution of the present invention contains stable electrons and super hydrating and wetting ability, and is capable of maintaining the body's pH in balance.

### BRIEF DESCRIPTION OF THE DRAWINGS

FIG. 1 shows the effects of Alka Vita on glucose, triglycerides, cholesterol, and body weight.

### DETAILED DESCRIPTION OF THE INVENTION

Alkalinity

Every living cell in a body creates waste products. All of these waste products must be discharged from the body if health is to be maintained. Virtually all waste products are acidic. However, for several reasons, the body cannot always rid itself of 100% of the waste products.

When the body has an excess of acid that it cannot neutralize, the acid will be stored in the body, mostly in the interstitial space, called the extracellular matrix (or the ground substance of the body). This matrix forms the connective tissue of the body, including collagen, bones, ligaments, tendons and muscles, as well at the fatty tissues that surround our organs.

When the pH of the body is too low, the body's ability to absorb vitamins, minerals and other nutrients is reduced. Acidic pH reduces energy production in the cells, such as the cell's ability to repair itself and to detoxify.

Chronic acidosis is believed to be an underlying contributor to diseases such as cancer, osteoporosis, insulin sensitivity leading to diabetes, and the number one killer in the United States, heart disease.

Alkaline materials neutralize acid. Consequently, ingesting alkaline water assists the body in dissolving and eliminating toxic acidic waste materials. Furthermore, increasing the body's pH enhances the oxygen concentration in the fluids by means of increased hydroxyl (OH—) ions. Indeed, blood with a pH of 7.15 contains almost 65% more oxygen than blood with a pH of 7.0.

Although alkaline water is not a drug to cure diseases, if consumed regularly, alkaline water will gradually reduce accumulated acid wastes.

Oxygen

Cancer cells live in an oxygen-free state (anaerobic) and are energy-deficient. Otto Warburg discovered decades ago that all cancer cells are anaerobic in their metabolism, in contrast with healthy cells that are mostly aerobic. Further, he showed that a healthy cell can easily be transformed into a cancerous cell by putting healthy cells in a Petri dish and removing all oxygen. The cells started to transform into cancerous, anaerobic cells very quickly.

Oxygen thus is crucial in inhibiting the proliferation of cancerous cells. Since cancer cells exist in an oxygen-free (anaerobic) state, they are energy deficient. This energy deficiency renders the cells unable to function normally, causing them to multiply uncontrollably. Fermentation of sugar (glucose) in cancerous cells replaces the respiration of oxygen found in healthy cells. Indeed, whereas healthy cells sustain energy levels with respiration of oxygen, cancerous cells meet their energy needs almost exclusively via fermentation. When the glucose contained in oxygen-deprived cells ferments, it creates acidity. The release of this toxic acidic waste into the body is believed to cause some of the pain associated with cancer.

Cancer, above all other diseases, has countless secondary causes. Almost anything can cause caners. However, even for cancer, there is one prime cause, the replacement of the respiration of oxygen in normal body cells by fermentation of sugar. All normal body cells meet their energy needs by respiration of oxygen, whereas cancer cells meet their energy needs largely by fermentation.

Thus, oxygen provides a crucial component in the present invention, counteracting and preventing the anaerobic environments conducive to proliferation of cancerous cells.

Sodium

The Electrolytes (Sodium, Potassium, Chloride)

The function of sodium, potassium, and chloride ions in the body are closely interrelated. Found in all body fluids, these electrolytes maintain the proper balance and distribution of fluids throughout the body.

Sodium, potassium and chloride are called electrolytes because in aqueous solution, such as in the body, they dissociate into charged ions, which contain either a positive or negative electric charge.

Silicon

Silicon stimulates and enhances immune function, and inhibits the aging of body tissues. An essential trace element, it is necessary for the development of normal bone and connective tissue. It appears to contribute to the development and construction of the bone's protein matrix, and may also increase the rate of bone mineralization and calcium deposition.

Silicon supplementation also shows promise in preventing atherosclerotic plaques, as demonstrated by experiments on rabbits at the University Pierre et Marie Curie in Paris.

Silicon also may counteract aluminum's toxic effect on the body, decreasing one's risk of developing Alzheimer's or osteoporosis. This is particularly important for elderly patients, as silicon levels tend to decrease with age.

Silicon proves useful in treating aging, Alzheimer's disease, cardiovascular disease, and osteoporosis. It enhances calcium absorption, heart health, immune function, skin and nail health, and heart health. Silicon aids in the deposition of calcium in the bones, and can assist in the remineralization of damaged bones. Additionally, by hardening the enamel of teeth, silica may help prevent cavities and preserve healthy teeth. It also prevents gum bleeding, atrophy, and recession.

Silicon is an essential trace element researchers believe is important to normal bond and connective tissue development. Unfortunately, modern food processing techniques strip our few remaining silicon-containing foods, such as grains and rice, of nearly all of their silicon content, thus depriving the body of the health benefits silicon provides.

Laboratory experiments on chicks and infant rats have demonstrated that silicon is essential for normal skeletal growth. Bone is a uniquely flexible material made of apatite crystals embedded in a protein matrix containing collagen and glycosaminoglycans. Silicon appears to play a role in the initial stages of bond development when the protein matrix is constructed. Silicon may also increase the rate of bone mineralization and enhance calcium deposition in bone, meaning that the bone grows faster and stronger.

Osteoporosis is a symptom of the aging process. As calcium is leached out of the bones, the bones become brittle and weak. Merely taking a calcium supplement cannot correct or stop this threatening and crippling disease, because the body cannot assimilate and use the calcium in the absence of silica. Evidence suggests that using only calcium, such as the world-wide advertised “Coral Calcium”, rather than adding to the calcium in the bones, rather leaches bone calcium, and thus hastens the degenerative process of osteoporosis and similar disease that affect the supportive and connective tissues in the human body.

For osteoporosis, silicon can stop the pain and even restore the body's self-repair process. Although osteoporosis symptoms attack women primarily after menopause, the degenerative process begins much earlier, when women are younger. More women die from fractures caused by osteoporosis than of cancer of the breast, cervix, and uterus, combined.

In order to re-mineralize damaged bones, it is recommended that a silicon supplement be taken daily. Bones are composed of mainly phosphorus, magnesium and calcium, but they also contain silicon. Silicon is responsible for depositing minerals into the bones, particularly calcium. Silicon speeds up the healing of fractures and also reduces scarring at the site of a fracture.

Tissue degeneration accelerates with aging, when connective develops an increasing inability to retain moisture. Silicon can help slow the degenerative process of connective tissue. With silicon, vitality and life, which are often lost in the later years, can be naturally maintained or even restored to the skin.

Hair is nature's greatest beauty enhancer. It can make people sexually attractive, and protects the body. Hair, which contains about 90 micrograms of silicon per gram, is almost as rich in silicon as are healthy bones. Silicon is a major component of hair. Silicon helps to prevent baldness, stimulates healthier hair growth, and assures beautiful shine, luster and strength.

By hardening the enamel, silicon prevents cavities and preserves teeth. Silicon also prevents bleeding gums, gum atrophy, and rescission that cases loosening of teeth.

Nails are complex protein structures that grow four to five millimeters per month, on average. In case of dietary deficiency, the rate of growth slows. Fingernails thus can be the first indicators of silicon deficiency. Demineralization of the nails precedes by far any decalcification of the bones. With silicon supplementation, fragile nails become normal within a short period of time. Silicon improves the harness of the nails, making them shinier and less prone to breaking.

The restorative effects of silicon are most noticeable on the hair, skin, nails and teeth. This is because the skin and hair require silicon essentially for the same purposes as other tissues.

The human body needs silicon, regardless of age. It is as important to provide the body with dietary sources of silica early in life, as it is during the aging process that silica levels in tissue usually drop off. Silicon has a direct influence on absorption of all minerals that the body requires to maintain health. Silicon adds to the quality of life and improves stamina and appearance.

Silicon guards against the degeneration of connective tissue that results from aging and an inability of the tissue to retain moisture. It enhances hair and nail strength when ingested consistently.

Silicon, the second most abundant element on earth, plays an important role in many body functions and has a direct relationship to mineral absorption, such as calcium absorption.

Silicon is one of the most important constituents of the body's connective tissue, including cartilage, arteries, tendons and ligaments. It functions as a cross-linking agent, providing strength, flexibility, and resilience to collagen and elastin connective tissues. It is known to play a part in the integrity of the bones, arterial walls, skin, teeth, gums, hair and nails, and has been used to alleviate eczema and psoriasis.

Osteoporosis is one symptom of the aging process. As calcium leaches from the bones, the bones become brittle and weak. Merely taking a calcium supplement cannot correct or stop this threatening and crippling disease, because the body cannot assimilate and make use of the calcium without the presence of silicon. Evidence suggests that using only calcium, such as the widely advertised “Coral Calcium”, does not affect healing of the bones. On the contrary, calcium alone accelerates the leaching of calcium from the bones, and thus hastens the degenerative process of osteoporosis and similar diseases that affect the supportive and connective tissues in the human body.

Silicon enhances the function of iron, calcium, magnesium, potassium and boron, and is essential for bone development and growth. Bones need silicon to re-calcify and to strengthen bone tissue. A silica deficiency in tissue causes a calcium deficiency, which in turn results in a loss of tissue elasticity.

For osteoporosis sufferers, silicon can stop the pain and even restore the body's self repair process. Although osteoporosis symptom attack women primarily after menopause, the degenerative process begin much earlier. More women are dying of complications resulting from fractures caused by osteoporosis than cancer of the breast, cervix and uterus combined.

For purposes of re-mineralization of damaged bones, it is recommended that a sufficient silicon supplement be taken daily. Bones are made primarily of phosphorus, magnesium and calcium, but they also contain silica. Silica is responsible for depositing minerals into the bones, primarily calcium. It speeds up healing of fractures and also reduces scarring at the site of a fracture.

Tissue degeneration accelerates due to aging when connective tissue develops an increasing inability to retain moisture. Silicon can help slow the degenerative process of connective tissue. With silica, vitality and life, which are often lost as the years accumulate, can be naturally maintained or even restored to the skin.

Hair is nature's greatest beauty enhancer. It makes people sexually attractive and serves to protect the head. Healthy hair, which has about 90 micrograms of silicon per gram of hair, is almost as rich in silica as are healthy bones. Silicon is a major component of hair. Silicon helps to prevent baldness, stimulates healthier hair growth, and assures beautiful shine, luster and strength.

By hardening the enamel, silicon prevents cavities and preserves teeth. Silicon also prevents bleeding gums, gum atrophy, and recession that causes the teeth to loosen.

Nails are complex protein structures that grow four to five millimeters per month, on average. In case of deficiency, the rate of growth slows. Therefore, fingernails can be the first indicators of silicon deficiency. Demineralization of the nails precedes by far any decalcification of bones. With silicon supplementation, fragile nails become normal within a short period of time. Silicon improves the hardness of nails, making them shinier and less prone to breaking.

People need silicon regardless of age. It is as important to give the body dietary silicon early in life as it is during the aging process, when silicon levels in tissues usually drop off. Silicon has a direct influence on absorption of all minerals that the body requires to maintain health. Silicon thus adds to the quality of life and improves stamina and appearance.

Many factors, including nutrition, hormones, excised, smoking, alcohol consumption, and genetics, play roles in osteoporosis and cardiovascular disease in humans. Preventing these chronic diseases may require a suite of nutrients, including silicon.

Hydrogen

Hydrogen plays a role in the majority of bodily processes, including the building and repair of immune function, organ systems, and cellular structures. Indeed, lack of hydrogen results in dehydration, which inhibits the assimilation of moisture and fatty nutrients. And causing abnormal nerve heat generation inside the body. The consequences of this impaired assimilation include brain shrinkage, face furrowing, dehydration of mucus, and tendon/nerve cramps. Lack of hydrogen also contributes to gout, muscular rheumatism, mental confusion, neck stiffness, irritated skin, and sore joints.

Other conditions cause by lack of hydrogen include gout, muscular rheumatism, mental confusion and inadequacy, neck stiffness, irritate skin and sore joints. This is why it is important to drink water, as most of our hydrogen comes from water, as well as fresh fruits and vegetables.

If the body does not rid itself of all of its waste products what then happens to those non-disposed acid waste products? The answer, of course, is basic chemistry. The waste products become solid wastes, such as excesses of cholesterol, fatty acids, uric acid, kidney stones, urates, phosphates, sulfates, etc. Unknown to a person, these waste products accumulate and build up within the body. This accumulation of non-disposed acid waste within the body accelerates the aging process.

Since alkaline substances neutralize acid, drinking alkaline water helps the body to dissolve acid wastes and makes it easier for the body to dispose of them. Alkaline water is not a medicine or drug to cure diseases. However, if consumed regularly, alkaline water gradually reduces the accumulated acid waste, producing a natural improvement in health.

Cancer cells live in an oxygen free state (anaerobic) and are therefore energy deficient. This finding can be traced back to 1920's science researcher, Nobel Prize winner Otto Warburg. With insufficient energy, the cell can no longer function normally, and it begins multiplying uncontrollably. The glucose of the oxygen-deprived cell ferments and becomes acidic, similar to the process in which meat and vegetables deteriorate and ferment. This toxicity thrives in an acidic environment. This release of toxicity into the body is believed to be the cause of pain associated with cancer.

Altering the toxic (acid) state of the cancer essentially eliminates the cancer cell's ability to live.

Cancer, more than any other disease, has countless secondary causes. Almost anything can cause cancer in sufficient amounts. However, even for cancer, there is one primary cause: the replacement of the respiration of oxygen in normal body cells by a fermentate of sugar. All normal body cells meet their energy needs by respirating oxygen, whereas cancer cells meet their energy needs in large part by fermentation.

### SUMMARY OF THE INVENTION

It is an object of the present invention to overcome the aforesaid deficiency in the prior art.

It is an object of the present invention to provide a composition that reduces or eliminates offensive odors of decomposition of organic matter.

It is another object of the present invention to provide a composition that increases the body's ability to resist disease.

It is a further object of the present invention to enhance the body's absorption of vitamins and minerals.

It is still another object of the present invention to provide a composition to maintain the pH balance of the body.

It is another object of the invention to provide a composition for hydrating and wetting.

It is a further object of the present invention to enhance the body's resistance to several types of cancer, HIV, hepatitis C, viruses, diabetes, acid reflux, upset stomach, yeast infection, genital herpes, and SARS.

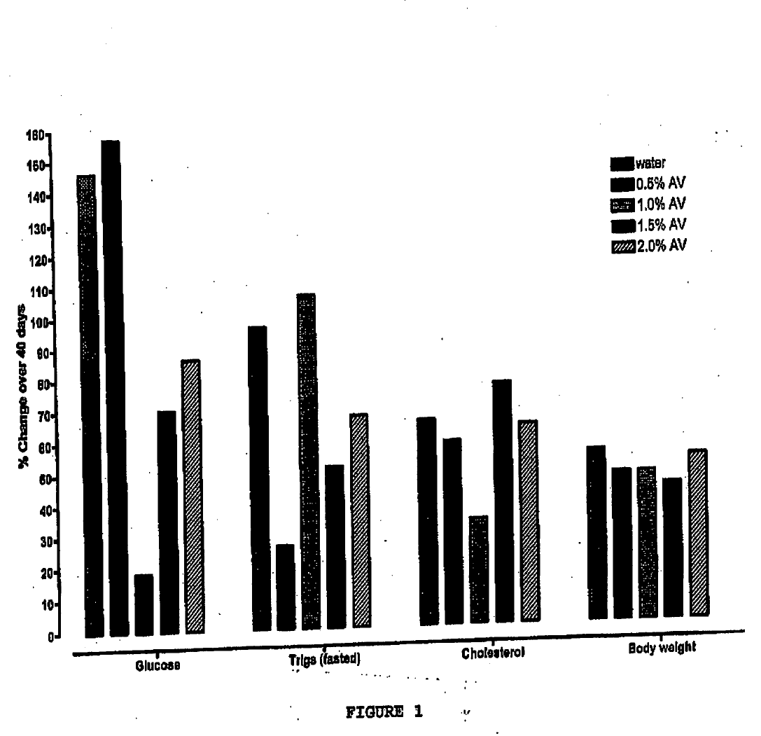

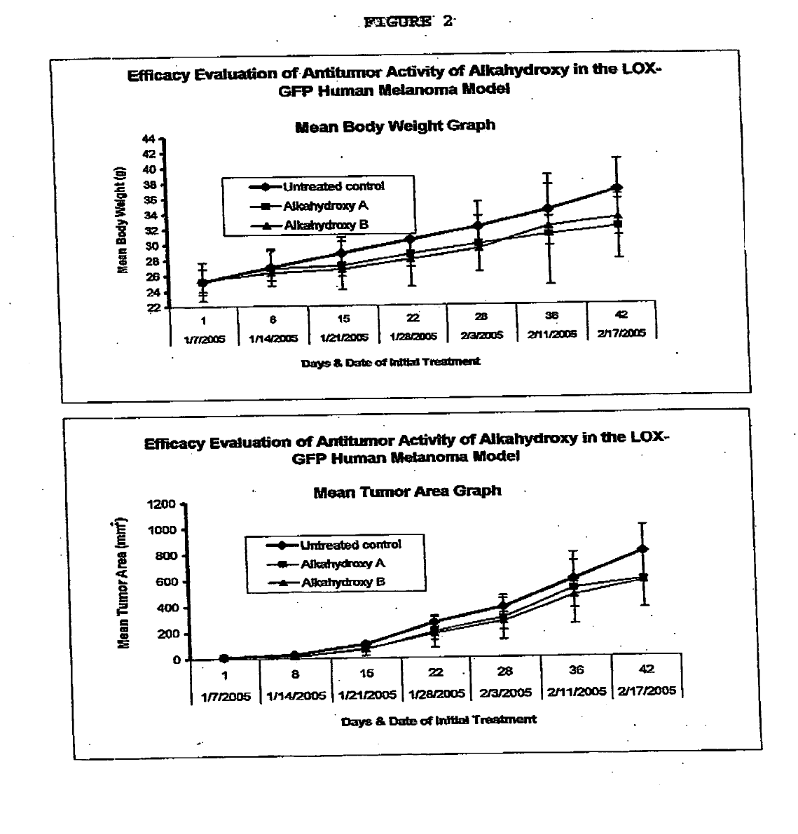

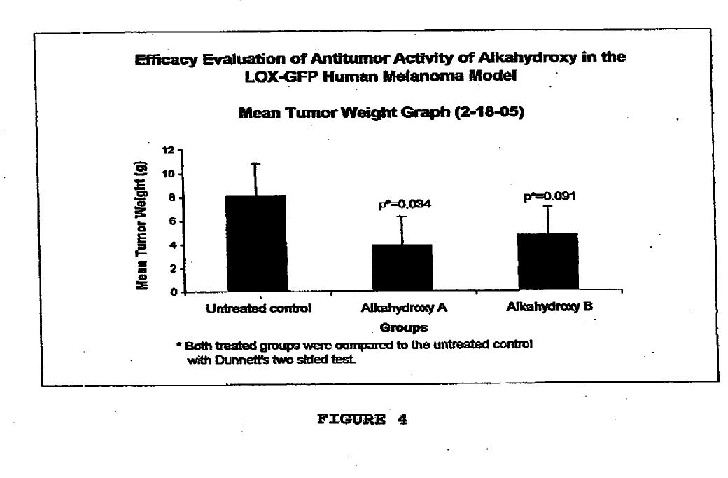

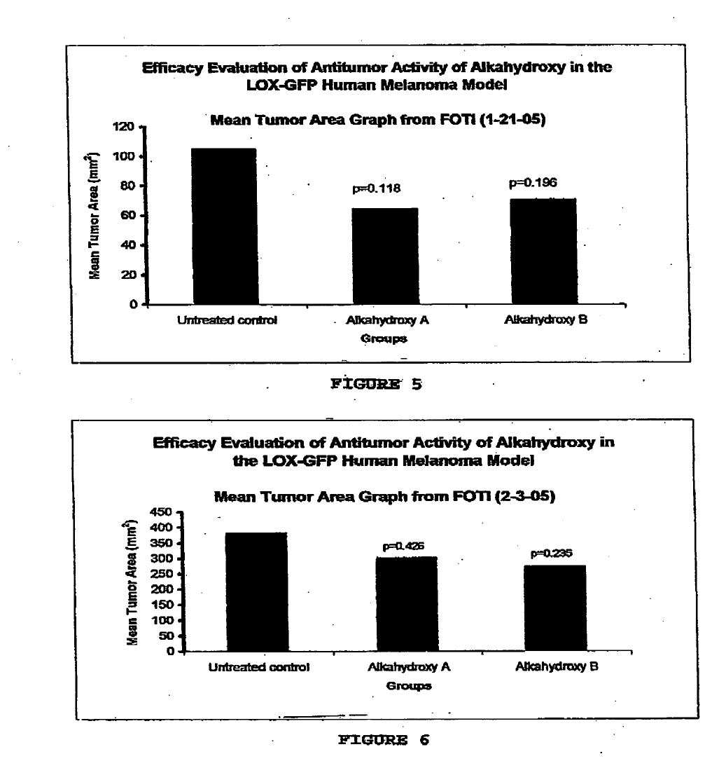

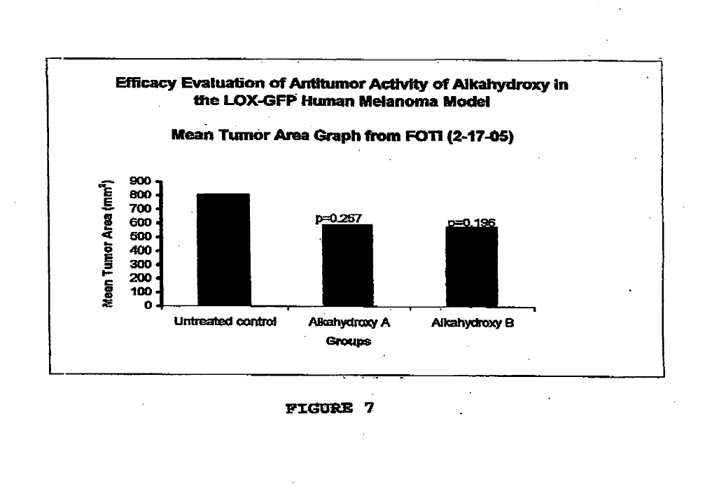

FIG. 1 is a graph showing the effect the alkaline solution of the present invention had on glucose, triglycerides, cholesterol, and body weight.

FIG. 2 is a mean body weight graph showing the antitumor activity of the alkaline solution of the present invention on LOX-GFP human melanoma model.

FIG. 3 is a mean tumor area graph showing the antitumor activity of the alkaline solution of the present invention on LOX-GFP human melanoma model.

FIG. 4 is a mean tumor weight graph showing the antitumor activity of the alkaline solution of the present invention on LOX-GFP human melanoma model.

FIG. 5 is a mean tumor area graph showing the antitumor activity of the alkaline solution of the present invention on LOX-GFP human melanoma model.

FIG. 6 is a mean tumor area graph showing the antitumor activity of the alkaline solution of the present invention on LOX-GFP human melanoma model.

FIG. 7 is a mean tumor area graph showing the antitumor activity of the alkaline solution of the present invention on LOX-GFP human melanoma model.

### DETAILED DESCRIPTION OF THE INVENTION

The alkaline water of the present invention, which contains at a minimum a soluble silicon compound, water, and a compound to raise the pH of the solution, can be used to maintain health, treat diseases such as cancer, heart disease, osteoporosis, diabetes, etc., and to prevent decomposition of organic matter such as fish and corpses. The alkaline water of the present invention easily and safely increases the body's pH to an optimal range, thereby addressing many of the health concerns of acidosis.

Additionally, the metal ions in the alkaline solution, which may be calcium, potassium, sodium, magnesium and iron, bind with acid minerals an evacuate acidic and toxic substances from the body. Consuming more alkaline foods and drinks allows for a mucousless body. A mucousless body has no sinus congestion, no chronic lymphatic congestion or swelling, no fluid accumulation in the lungs, no joint calcification, no calcium deficiency, and many other benefits. A person whose pH is properly balanced will be happy, content, free of body aches and muscle cramps. These persons enjoy being emotionally stable and mentally clear. The results are extraordinary because the tissues exist in their natural and optimal alkaline environment, which is conducive to good health.

Alkalinity has a number of important benefits for the body, including:
a. creating a sense of oneness and harmony;
b. encouraging the growth of friendly bacteria;
c. maintaining healthy organs and glands;
d. minimizing bodily aches;
e. diminishing illness;
f. producing relaxed behavior;
g. producing a mucousless body;
h. enhancing cellular health;
i. producing well-being and happiness.

The following examples are case studies of patients treated with the composition of the present invention. In the following examples, the composition of the invention is referred to an Alka Vita.

In all of the following examples, the composition contained 1% silicon (0.057 mg/l) and 0.04 mg/l sodium in water. The minimum dosage fore these example is one ounce per day. The maximum dosage is four ounces per day.

### EXAMPLE 1

In March, 1997, a 54 year old male had 14 lymph nodes removed from each side of the prostate. Several of the nodes on the left side were positive, none on the right side were positive.

In April, 1997, 25 radiation treatments were administered to the patient.

May, 1997, radioactive seeds were installed inside the prostate. The seeds were removed 26 hours later.

July, 1997, the psa began to be elevated. The patient was placed on Lupron and Casodex. 1998, the patient began having pain in the back in the area of the ninth rib that would awaken him at night. The patient learned to live with the pain.

Mar. 19, 2002, the patient stopped taking the Casodex and started taking Alka Vita twice a day on an empty stomach. The back pain, which he had suffered since 1998, was completely gone by Mar. 23, 2002. His wife and other family members noticed that the patient's skin color had returned to the color is was prior to the cancer.

Apr. 29, 2002, the patient went for his two month physical. The patient's test results showed that all conditions were perfectly normal, with no trace of cancer. The patient informed the physician that he had discontinued the Casodex and began taking a liquid the contents of which were unknown to him, only that it was supposed to raise the pH of his body to 7. The physician told the patient that with the test results just obtained and the physician's belief that the pH of the body should be maintained at a pH of at least 7, that whatever the patient was taking should be continued, and all other medications should be stopped.

May, 2002, large scale testing was undertaken for a total body bone scan and a cat scan of the pelvic area, along with twelve X-rays of the rib cage. The result was that no cancer was found. This patient's father died of cancer, and the patient's mother, wife, and other family members had believed that the patient was dying of cancer.

August, 2002, the patient's psa level as 0.2, whereas at the beginning of the cancer treatments in 1997 his psa was 91.3. Six months ago the patient's cholesterol was 277, and was then 220.

May, 2003, the patient is still in good health.

### EXAMPLE 2

A 44 year old female, diabetic, was diagnosed with stage 2 ovarian cancer, and a tumor begun growing outside her uterus. November, 2001, the tumor was the size of a golf ball. By the first week of February 2002, the tumor was the size of a soccer ball and growing. The patient was also told that cancer cells had been found in the uterus. The patient was in constant pain, unable to sleep, suffering from migraine headaches and severe acid indigestion. She had severe weakness in the legs and had fallen several times. She was told that, even with the tumor excised, she had six months to live.

The patient began douching with Alka Vita twice a day on an empty stomach. After that her pain was gone, and her physician informed her after an examination that the tumor appeared to be shrinking. A Pap smear showed no evidence of cancer in the uterus. The physicians were totally surprised and have recommended a series of additional tests to verify the result. In addition, although the patient had been taking insulin for yeas, her blood sugar had begun to decrease.

May, 2002, the patient reported that she had vaginal itching and douched with Alka Vita that day and for two mornings thereafter. The afternoon of the third day, she used the bathroom and felt herself pass something vaginally, which was a mass approximately 1 inches in size. She stated that the previous November she had felt a constant ache in that area, and, since passing the mass, she no longer feels any aching.

The patient further explained that with her physical problems she has not been able to have a fulfilling sex life and this, along with other physical problems, had led her to suffer from severe depression and even contemplate suicide. She then related that she had achieved sexual fulfillment for the first time in about a year, and that she “felt like a woman again.”

June, 2002, the patient is in good health and recently returned from the first week-long vacation in many years.

August, 2002, the patient continues to improve overall, feels good, and looks good.

### EXAMPLE 3

A male with severe pain and infection following a root canal began rinsing his mouth with Alka Vita for two days. After two days, the pain was almost completely gone, and the swelling from the infection disappeared the second day.

### EXAMPLE 4

A 21 year old male and a 24 year old female both had a severe case of acne vulgaris and skin problems on the face. They had both tried every medication prescribed by physicians. They began using a lotion prepared with Alka Vita as a base.

### EXAMPLE 5

A 23 year old female had suffered from a yeast infection for 1.5 years. She had tried every medication and antibiotics that the physicians had prescribed, but to no avail. She began using Alka Vita as a douche with warm water. Three days later the infection had disappeared.

### EXAMPLE 6

A 76 year old male was diagnosed with cancer of the bladder and given five to six months to live. Kensiology and viral tests were abnormal. The patient began taking Alka Vita. Three and one half months later the patient went for a complete physical, and the physicians informed him that they could not find a trace of cancer.

### EXAMPLE 7

A 38 year old female had vaginal discharge with strong odor and strong bad breath. She douched three times with Alka Vita and drank one ounce of Alka Vita per day. After seven days, the vaginal disgorge and the mouth odors had disappeared. Several months later the problems had not recurred.

### EXAMPLE 8

A 43 year old female complained that the bones in her legs ached constantly, and she also suffered from shortness of breath, vaginal odor, and discharge. The patient had taken Halen (fermented soy) along with nutrients with some results, but the symptoms always recurred. The patient began taking Alka Vita Feb. 10, 2002, once a day on an empty stomach. The ache in the bones in the lower extremities disappeared within six days. The patient then douched for two days and the vaginal discharge and odor disappeared. Six months later the problems had not recurred.

### EXAMPLE 9

A 20 year old male had skin discoloration on the arms along with some STD problems. He began taking Alka Vita. The skin cleared up within three weeks, and the STD problem cleared up after two weeks.

### EXAMPLE 10

A 70 year old female visited a dentist with severe mouth pain. The dentist prescribed antibiotics for an infection, but the patient had no relief. The patient began using Alka Vita as a mouthwash. The first and second day she experienced a slight burning sensation. The third day there was less burning and the patient felt a tightening of her teeth and gums. By the fourth day, there was no burning and a better feeling in the mouth. By the fifth day the infection had subsided.

### EXAMPLE 11

A 40 year old female smokes heavily and has bad breath. The tongue and palate were sprayed with Alka Vita for 2-3 minutes. The breath odor was neutralized. The patient also had been diagnosed with a herniated disk which affected her right shoulder and arm. The patient began drinking Alka Vita with juice once a day. The patient feels better. Sore on her neck and back were sprayed with the composition and the sores disappeared.

### EXAMPLE 12

A 48 year old female had acne vulgaris on the lower part of the chin. The patient used Alka Vita undiluted, and the skin cleared up in two days. Noticeable results were seen within 6 to 12 hours. The patient also treated a sore that had become infected with the product, and the sore disappeared.

This patient had been in the hospital in January, 2002, with pneumonia, and was taking antibiotics. As is not uncommon with women taking antibiotics, she developed a white discharge commonly associated with a yeast infection. The patient douched three days with Alka Vita. The first day the patient experienced a burning sensation, similar to alcohol, with odor present. The second day there was no burning but the odor was still present. The third day there was no burning or odor.

The patient used Alka Vita as a facial cleanser. The composting was applied under the eyes, nose and cheeks. It cleaned the skin very thoroughly, and left the face soft when washed off, with a feeling of skin toning.

This patient also had a painful tooth infection. She held Alka Vita compounds of the present invention in her mouth for three minutes. The first day she experienced a burning sensation. The second day there was less burning and less pain. By the third day, the infection and the pain had disappeared.

Six months later, the patient continues to fell good. She has used the Alka Vita full strength on cuts and wounds, which healed with just one treatment.

### EXAMPLE 13

A 43 year old male had suffered from acid reflux for two years. The reflux was severe enough to wake him once or twice every night, and he slept in an inclined bed. The patient has tried Propulsid, Pepcid AC, Zantac, Prilosec and Nexium, with no relief. The patient drank a small glass of water with Alka Vita each night. From the first night the patient began taking the Alka Vita and every night thereafter there is no more acid reflux. Nine months later, the acid reflux did not return and the patient no longer sleeps in an inclined bed.

### EXAMPLE 14

A 55 year old male was diagnosed with throat cancer, and three physicians gave him six months to one year to live. He had lost much weight and had a large inflamed growth on one side of his face that was hard to the touch. The patient began taking two ounces of a 2% solution of the Alka Vita invention in July, 2002. Within one month, the constant headache and pain had had before was considerably reduced. The growth was reduced in size and it was now soft to the touch. The patient has started gaining weight. And reports considerable overall improvement.

### EXAMPLE 15

A 43 year old male had been a diabetic for 20 years. The patient checks his glucose level daily. The patient began taking the composition of the present invention once in the morning daily and his glucose level dropped. His glucose level remains between 80 and 85.

### EXAMPLE 16

A female had been diabetic for several years, with a glucose level of 240. The patient began taking Alka Vita in the morning and in the evening at the beginning of July. One month later, her glucose level was 80. The patient now takes Alka Vita once in the evening every other day, and her glucose level remains at 80.

### EXAMPLE 17

A 43 year old female had been suffering from multiple sclerosis for many years. She began drinking Alka Vita once per day on an empty stomach, and at the end of the first month she began having feelings in the tips of her fingers. Improvement continued, and the patient eventually developed feelings in the entire finger.

### EXAMPLE 18

A 33 year old female had suffered from ulcerative colitis for a very long time, and surgery had been recommended. The patient began colonics and enemas for one week, and applied a vaginal pad soaked in Alka Vita. She retained eth pad with the Alka Vita in place for as long as possible. The first treatment caused a small burning sensation, but the burning diminished with each additional treatment. She returned to her physician for another evaluation, and the physician informed her that her ulcers were healing and there was no longer a need for surgery.

### EXAMPLE 19

A 46 year old female began having muscle spasms 29 years previously, just prior to the birth of her daughter. Years later these spasms became epileptic seizures. In the past four years, this patient has had a minimum of two and a maximum of four seizures per day. The patient began taking Alka Vita at the end of November. Several months later, she has since had two seizures. She claims that she feels good, and her complexion has improved. She has a better disposition and doe not feel angry and nasty all the time.

### EXAMPLE 20

Sixteen months previously, a 60 year old male suffered a stroke. For many years, he had poor circulation and varicose veins, and had a constant pin on the left shoulder and in his legs. Now, after taking Alka Vita, he can sleep on his left shoulder without pain and the pain in his legs has disappeared.

### EXAMPLE 21

A 91 year old male had diabetes and high blood pressure for many years. He used to walk every day in the local park, but had to stop because he no longer had the strength to walk. HE began taking Alka Vita. Within 40 days his glucose level was below 100, his blood pressure is near normal, and he has returned to walking in the park every day.

### EXAMPLE 22

A 48 year old male, 6′1″ and weighing 366 pounds, was diagnosed with Type 2 (NIDDM) diabetes in 1987. For 16 years he had been taking Diabeta for control of the condition. The dosage has ranged from 10 mg/day to 20 mg/day.

In June, 2002, the patient was hospitalized for surgery on the right foot. Following surgery, the patient was recliner bound for twelve weeks and gained approximately 40 pounds. The blood glucose level over that time period went from about 130 (morning-fasting) to over 200. in early November, I was prescribed Adventia in addition to the 20 mg/day of Diabeta. The patient's cholesterol level was over 320 at that time, and triglycerides were 600.

The patient began taking Alka Vita two weeks prior to Thanksgiving, 2002. Within one week, the blood glucose had dropped from about 220 to about 150. After a little over a month of taking Alka Vita, the blood glucose was in the 120-130 range. The December blood test showed cholesterol down to 255 and triglycerides down from 600 to 423.

By January, 2003, the patient's blood glucose had been reduced to 80-100 (morning-fasting), and the patient can now eat late at night, even ice cream on occasions, and the morning glucose is normal.

This patient also had an acid reflux problem for a long time. Almost immediately after taking Alka vita, the acid reflux disappeared and did not return.

The dosage is two ounces of Alka Vita with 2-4 ounces of white grape juice (for taste), twice a day, on an empty stomach.

January, 2003, after watching the Super Bowl, the patient ate a considerable amount of food, including ice cream. The next morning the fasting glucose was 89. The patient has reduced his insulin intake and begun taking Alka vita once per day. By May, 2003, the patient required only one dose of insulin daily.

Why Alkaline Water can Decrease Diabetic Peripheral Neuropathy and Other Myelin Deficiency Disorders

Microvascular vasodilation may be due to localized release of nitric oxide (NO) from red blood cells. NO activates guanylate cyclase in smooth muscle cells and, after formation of cGMP, phosphorylation of myosin occurs. Additionally, this myosin activation also phsophorylates the potassium channel via cGMP. This latter event is the primary cause of NO mediated vascular smooth muscle cell relaxation and the parallel increase in circulation.

Diabetic patients cannot produce NO at the same rate or to the same degree as non-diabetic subjects. Not only is the activity of the enzyme that generates NO from L-arginine defective (possibly due to metabolic acidosis that attends diabetes), but glycosylated hemoglobin, characteristic of diabetes, avidly binds any NO that is formed. This latter constraint suggests that even the small amounts of NO produced in diabetics may not be easily released from red blood cells at the microcirculatory sites. In addition, blood glucose binds NO and therefore, the hyperglycemia of diabetes would also be expected to limit NO bioavailability at the microcirculatory level. Diabetic patients, therefore, are probably not able to produce or release normal amounts of NO. Impaired regulation of local blood flow and the accompanying reduction in nutrition and oxygenation of peripheral tissues, including nerves, might be partly responsible for the symptoms of diabetic peripheral neuropathy. However, if metabolic acidosis can be mediated by the introduction of Alka Vita, the red blood cells would be able to more readily store higher levels of NO in the form of nitrosothiols, which would then have more bioavailability at the microcirculatory sties.

### EXAMPLE 23

A 35 year old female was diagnosed with ovarian cancer Oct. 2, 2002. The tumor was 11.8×8.1×9.0 cm. The patient had a hysterectomy Oct. 7, 2002, and the tumor was removed. The estimate was that 5% of the cancer was left.

The next day, Oct. 8, 2002, the patient began drinking Alka Vita once per day in the morning. She also began taking some herbal remedies. She was programmed to have six chemotherapies every three weeks, platinol and taxol. Before she began the first chemotherapy, just by her taking the Alka Vita, the size of the tumor dropped from Ca 125 237 μ/ml to Ca 125 132 μ/ml. Since that time the patient has continued taking Alka Vita.

After the second chemotherapy, the patient began taking Alka vita twice a day. At the end of the chemotherapy treatment the Ca 125 wend from 132 to 30.98 U/ml. With those results, the physician conducted a tomography test. The results showed no residual cancer. A second laparotomy operation was conducted, and found that the spleen was clean and the peritoneal area contained a reddish tissue of 0.2 cm×0.3 cm in diameter.

### EXAMPLE 24

May 1, 2003, a 61 year old female had breast cancer, which was treated with a mastectomy and two rounds of chemotherapy. At that point the cancer cells were still viable. The patient's blood profile showed that the immune system was compromised and very toxic. The patient was administered Alka Vita 1 ounce daily, with juice. The patient also used Alka Vita as a daily mouthwash, for mouth odor. The patient was also given IV drips weekly along with colonics. Kensiology exam showed fungal, bacteria, and viral infections.

Jun. 18, 2003, kensiology showed only half of the previous fungal, bacterial and viral infection. The patient reported feeling better.

Jul. 21, 2003, the patient was again tested, and only 5 to 6 vials showed on kensiology testing. The patient greatly improved, and no active cancer cells were found. The patient was left on maintenance protocol of blood assessment every four months, monthly colonic and IV drips, and one ounce of Alka Vita daily.

### EXAMPLE 25

On Mar. 3, 2003, an 81 year old male was diagnosed with enlarged prostate with tumor. The patient wanted neither radiation nor surgery. A blood assessment and kensiology were conducted. The results showed viral, fungal and bacterial infection, very toxic. The patient was given Alka Vita daily with juice, IV therapy, colon hydrotherapy and supplement therapy.

Apr. 9, 2003, the patient reported feeling better. Kensiology testing revealed less vials on fungal, bacterial, and viral infection. The therapy was continued.

May 13, 2003, the patient reported doing well. Blood testing and kensiology testing showed significant improvement. The IV therapy was increased to weekly, Alka Vita increased to 1 ounce twice daily, colon hydrotherapy weekly.

Jun. 11, 2003, the patient returned from M.D. Anderson Hospital. Testing there showed no active cancer cells. The patient was left on a regime of IV and colon therapy along with Alka Vita and nutritional supplements. Testing was continued every four months.

### EXAMPLE 26

Mar. 6, 2003, a 48 year old patient presented severe chronic symptoms of fibromyalgia. Blood assessment and kensiology testing were conducted, revealing fungal, bacterial and viral infections. The patient was give Alka Vita one ounce daily with juice, IV therapy, colon therapy, steam therapy, and nutritional supplements.

Apr. 10, 2003, the patient reported feeling better, and the same regimen was continued. Kensiology testing showed improvement in that some vials were down.

May 8, 2003, the patent reported feeling better than she has in years. The patient still showed fungal and viral infections, but les than in the previous month. The therapy was continued.

Jun. 12, 2003, the patient was doing well, and therapy decreased to two IV per month, one week Alka vita per month, colon therapy every other month.

Jul. 9, 2003, the patient was still doing well. The regimen was decreased to one IV monthly, nutritional supplements, and Alka Vita one week, monthly.

### EXAMPLE 27

A 56 year old male was admitted with a severe infection in the right leg. The patient had been seeing other physicians for the last four months, but his condition continued to worsen. Blood and kensiology testing were conducted, and showed that the patient had viral, fungal and bacterial infection and malabsorption of nutrients. The patient was prescribed IV therapy, Alka Vita two ounces twice daily, Alka Vita sprayed onto the wound area, and colon therapy.

Jun. 17, 2003 the patient showed improvement. The le was not as red as previously, and some bands of normal skin color returning. IV therapy was continued, along with spraying eth wound area with Alka Vita three to four times daily, and ingesting 1 ounce Alka Vita twice daily. Colon therapy was continued twice weekly, and a nutritional supplement was added.

Jul. 9, 2003, the patient was doing better and the leg had more normal flesh color. The above treatment was continued.

### EXAMPLE 28

Apr. 3, 2000, a male patient came for treatment of a chronic sinus infection. The patient was not able to gain weight. Blood and kensiology assessment showed viral, fungal, and bacterial infection. The patient was given IV therapy, Alka Vita to drink one ounce daily, and an alkaline nasal wash to use three to four times daily.

May 13, 2003, the patient was feeling better. The patient agreed to two days of IV therapy and continuation of the above regiment.

Jul. 20, 2003, the patient was feeling better and the sinus area looked much better. The patient was put on a regimen of Alka Vita one ounce daily for two weeks per month and nasal wash when needed. IV therapy was reduced to once monthly. Nutritional supplements were recommended.

### EXAMPLE 29

Jan. 16, 2003, a 32 year old female who had been tested by gynecologists for endometriosis was assessed with blood and kensiology test. These test revealed a viral, fungal, and bacterial infection. The patient was given Alka Vita to drink one ounce daily, and to douche two to three times a week with Alka Vita diluted to 1.5 ounce with distilled water. The patient was also given Alka Vita to use as a mouth wash for mouth order, along with IV therapy and colon therapy.

Feb. 11, 2003 the patient was feeling somewhat better. The vaginal area was tender, and there was some burning with the douche. The regimen was continued.

Mar. 10, 2003, the patient felt better. The vaginal area was no longer tender, and the douche water ran more clear than before. Douching was conducted twice weekly at 3% strength. The remained of the above regimen was continued.

Apr. 16, 2003, the patient was feeling better. Tests conducted for endometriosis found none. The patient went on a maintenance program of IB therapy, colon therapy, and Alka Vita 1 ounce daily for one week every month, alkaline douche twice monthly.

May 14, 2003, the patient reported no abdominal pain. The patient continued the maintenance program.

### EXAMPLE 30

January 8, 203, a 48 year old female patient came in with chronic urinary tract infection. The patient was instructed to drink one ounce Alka Vita daily, and alkaline douches three to four times weekly.

Feb. 14, 2003, the patient was feeling better and had not had recurrent urinary tract infection. The patient was advised to continue douching weekly.

Mar. 18, 2003, the patient was still doing well, with no evidence of urinary tract infection. The above regimen was continued.

Apr. 16, 2003, the patient was still doing well and remained on the program for maintenance.

### EXAMPLE 31

A 38 year old male came in with chronic mouth ulcers. Alka Vita was prescribed daily ½ ounce with ½ ounce distilled water to reduce the burning sensation and also to use as a mouth wash.

Feb. 11, 2003, the patient cane in feeling better, and was able to take 3% Alka Vita solution without dilution. Most of the ulcers were gone.

Feb. 18, 2003, the patient's mouth had no sores.

### EXAMPLE 32

A 48 year old male presented caner in the left upper thigh that had spread into the bones and the lungs. Surgery had been performed five years previously to remove thigh cancer. By 2002 the cancer had spread to the bonds and lungs.

Dec. 30, 2002, the patient began drinking Alka Vita. After one month the face began to look healthier, but the patient did not maintain treatment with Alka Vita.

Jun. 30, 2003, the patient was ill again, and stated that muscle cancers were found near the right armpit and back, which were surgically removed. The patient has resumed drinking Alka Vita.

### EXAMPLE 33

A 48 year old male was suffering from kidney cancer which had spread into his lungs. Surgery was conducted to remove one kidney. Because of chemotherapy and radiation treatment the patient had post hair, his face was pale, and the patient was barely able to walk and talk.

Oct. 10, 2003, the patient began drinking Alka Vita. After one month his face was healthy looking, the patient reported feeling better, and he returned to work. After three months of taking Alka Vita, a CT scan reveled no more progress of the cancer.

Jul. 3, 2003, the patient had been taking Alka Vita for seven months and reported feeling great. Full testing was scheduled for the end of August, 2003.

Jul. 30, 2003, the patient remains in good health.

### EXAMPLE 34

A 33 year old woman had breast cancer which had spread into the bones, pelvis and shoulder bone. Surgery was conducted at Samsung Hospital, followed by chemotherapy and radiation treatment. The patient was unable to move at all and had to be fed by family members. The treatments had caused a burning pain in the esophagus and stomach, and the patient was barely able to eat.

Mar. 17, 2003, after the first drink of Alka Vita the patient reported that her throat pain was reduced.

Apr. 17, 2003, the patient's condition was improving.

May 17, 2003, the patient was walking unaided for 30 minutes a day.

Jun. 17, 2003, the patient was able to travel 400 kilometers by car to attend a meeting.

Jul. 3, 2003, the patient was driving by herself, and was continuing to improve.

Jul. 4, 2003, the MRI and CT scan showed a spread of the cancer. These results are confusing, because the patient feels better and is not in pain. The patient continues to take Alka Vita and will continue to monitor her condition.

### EXAMPLE 35

A female, 49 years old, had breast cancer which began ten years previously. The patient has returned to hospital three times and the cancer has now spread throughout her body, despite repeated chemotherapy and radiation treatment. The patient ahs managed to continue working the entire time, even during the chemotherapy and radiation treatments.

April, 2003, the patient began taking Alka Vita along with her prescription medicines and chemotherapy treatments. The patient reported less fatigue, less hair loss, and more rapid recovery from chemotherapy treatments. Since beginning treatment with Alka Vita she no longer has headaches after chemotherapy.

### EXAMPLE 36

A 63 year old patient developed breast cancer five years ago, which then moved to the right breast and left arm. She was also diagnosed with Paget's disease. Her chest is blackish and hard, with dark red coloring and streaks. The left arm was thick and hard, very swollen. The patient was unable to bend the arm or her fingers. The cancer was considered to be untreatable by her physicians, and the patient entered Metro Hospice for pain management and to await death. Her roommate had exhibited improvement upon taking Alka Vita, so this patient requested to try it as well.

Jul. 10, 2003, the patient can now make a fist with her left hand and bend her left arm. She is now able to wash her face using her left arm and hand. In addition to drinking Alka Vita, she is now using it as a spray on her chest and arm.

Jul. 16, 2003, the left armpit and left arm are much less swollen than previously, and the patient can now comfortably wear a robe.

Jul. 18, 2003, the patient was released from Metro Hospice and returned home.

Aug. 7, 2003, the patient's right hand looks almost normal and her arm and armpit area are much thinner. She is beginning to feel warmth in the lower arm. The patient continues to take 500 cc of Alka Vita twice daily, plus a spray of 3% Alka Vita.

### EXAMPLE 37

A 71 year old male was suffering from acute tuberculous bacillus endocarditis and produces abdominal dropsy. The patient was treated by injection and suction, but this caused stiffness of the endocardium, resulting in a fast heart rate and difficulty in breathing. The patient was unable to wake even short distances, and his condition worsened with time.

Jun. 23, 2003, the patient began taking 20 cc of Alka Vita twice daily.

Jul. 16, 2003, the patient reported feeling more comfortable breathing. He has been able to walk longer distances and has walked by himself for the first time in several months.

Aug. 1, 2003, the heart rate dropped form 150 to 70, and the patient continues to breathe easily.

### EXAMPLE 38

A 67 year old male with Type II diabetes requires insulin injections and pills daily.

Jun. 4, 2003, the patient began drinking 12-13 cc of Alka Vita daily.

Jul. 10, 2003, the patient's blood glucose dropped to around 140.

Jul. 30, 2003, the patient maintains the blood glucose level at 140, and continuing with insulin injections.

### EXAMPLE 39

A 65 year old female had Type II diabetes for eleven years, requiring insulin injections and pills daily. By 10:30 AM extreme fatigue set in and the patient was unable to do anything for the remainder of the day. There was not much feeling in the finger tips, and the patients were unable to hold a glass or cup well.

May 10, 2003, the patient began drinking 7 cc of Alka Vita daily.

May 13, 2003, the patient called to report that she is no longer tired and states that she now has feelings in the tips of her fingers and can now hold a cup properly and securely.

May 31, 2003, the patient has on her own discontinued insulin injections. She said that she had a problem of phlegm every morning, and this has now stopped.

Jul. 10, 2003, the patient continues to be able to work very hard around her farm without exhaustion.

Jul. 29, 2003, the patient states that she continues to improve.

Alkahydroxy A was 1% silicon solution, Alkahydroxy B was 2% silicon solution.

Animal studies were conducted to ascertain the effects Alka Vita has on blood glucose, cholesterol, triglycerides, and weight. Groups of rats were administered 2.0% Alka Vita, 1.5% Alka Vita, 1.0% Alka Vita, and 0.5% Alka Vita. The control group was give water. It can be seen from Tables 1-5 and FIG. 1 that Alka Vita was most effective in treating blood glucose when administered at 1% Alka Vita; in treating triglycerides, when administered at 0.5% Alka Vita; cholesterol, when administered at 12.0% Alka Vita. Weight gain was most improved with water of 2% Alka Vita.

TABLE 1

| Date of Study: | | 11 Aug-21 Sep 2004 | | | | | | | | | | | | |
|---|---|---|---|---|---|---|---|---|---|---|---|---|---|---|
| Animals Used in Study: | | ZDF-obese males | | | | | | | | | | | | |
| Date of Birth: | | 17-Jun-04 | | | | | | | | | | | | |
| | | Glucose (mg/dl) | | | | | | | | | | | | |
| | | 12-Aug | | | | | 13-Aug | | | 23-Aug | 30-Aug | 7-Sep | 13-Sep | 20-Sep |
| | ANIMAL | 0 | 2 | 4 | 6 | 8 | 24 | 16-Aug | 18-Aug | DAY | DAY | DAY | DAY | DAY |
| | ID | TIME | HOURS | HOURS | HOURS | HOURS | HOURS | DAY 5 | DAY 7 | 12 | 19 | 28 | 34 | 41 |
| Group 1 - | 101 | 165 | 164 | 163 | 125 | 127 | 146 | 128 | 125 | 195 | 189 | 389 | 435 | 473 |
| RO | 102 | 243 | 195 | 147 | 191 | 179 | 205 | 162 | 163 | 269 | 328 | 485 | 531 | 533 |
| H2O | 103 | 214 | 203 | 203 | 175 | 206 | 170 | 199 | 161 | 211 | 169 | 376 | 419 | 522 |
| | MEAN | 207.33 | 187.33 | 171.00 | 163.67 | 170.67 | 173.67 | 163.00 | 149.67 | 225.00 | 228.67 | 416.67 | 461.67 | 509.33 |
| | ST DEV | 39.43 | 20.60 | 28.84 | 34.43 | 40.15 | 29.67 | 35.51 | 21.39 | 38.94 | 86.60 | 59.53 | 60.58 | 31.94 |
| | SEM | 27.88 | 14.57 | 20.40 | 24.34 | 28.39 | 20.98 | 25.11 | 15.12 | 27.53 | 61.24 | 42.10 | 42.83 | 22.59 |
| Group 2 - | 104 | 163 | 197 | 186 | 177 | 221 | 170 | 151 | 148 | 221 | 317 | 488 | 509 | 557 |
| 0.5% | 105 | 333 | 310 | 250 | 159 | 200 | 337 | 208 | 176 | 356 | 386 | 506 | 522 | 554 |
| Alka Vita | 106 | 143 | 136 | 155 | 159 | 131 | 154 | 139 | 140 | 255 | 304 | 456 | 488 | 530 |
| | MEAN | 213.00 | 214.33 | 197.00 | 165.00 | 184.00 | 220.33 | 166.00 | 154.67 | 277.33 | 335.67 | 483.33 | 506.33 | 547.00 |
| | ST DEV | 104.40 | 88.29 | 48.45 | 10.39 | 47.09 | 101.35 | 36.86 | 18.90 | 70.22 | 44.07 | 25.32 | 17.16 | 14.80 |
| | SEM | 73.82 | 62.43 | 34.26 | 7.35 | 33.29 | 71.67 | 26.07 | 13.37 | 49.65 | 31.16 | 17.91 | 12.13 | 10.46 |
| Group 3 - | 107 | 231 | 215 | 221 | 173 | 201 | 218 | 157 | 144 | 195 | 113 | 152 | 157 | 195 |
| 1.0% | 108 | 126 | 111 | 117 | 129 | 147 | 129 | 127 | 134 | 137 | 125 | 144 | 133 | 144 |
| Alka Vita | 109 | 157 | 140 | 131 | 136 | 146 | 184 | 146 | 143 | 140 | 149 | 187 | 181 | 270 |
| | MEAN | 171.33 | 155.33 | 156.33 | 146.00 | 164.67 | 177.00 | 143.33 | 140.33 | 157.33 | 129.00 | 161.00 | 157.00 | 203.00 |
| | ST DEV | 53.95 | 53.67 | 56.44 | 23.64 | 31.47 | 44.91 | 15.18 | 5.51 | 32.65 | 18.33 | 22.87 | 24.00 | 63.38 |
| | SEM | 38.15 | 37.95 | 39.91 | 16.72 | 22.25 | 31.76 | 10.73 | 3.89 | 23.09 | 12.96 | 16.17 | 16.97 | 44.82 |
| Group 4 - | 110 | 130 | 152 | 134 | 136 | 154 | 156 | 135 | 130 | 143 | 153 | 159 | 167 | 209 |
| 1.5% | 111 | 293 | 254 | 179 | 212 | 211 | 249 | 186 | 311 | 397 | 440 | 558 | 576 | 583 |
| Alka Vita | 112 | 415 | 386 | 379 | 422 | 386 | 386 | 418 | 440 | 493 | 526 | 573 | 555 | 636 |
| | MEAN | 279.33 | 264.00 | 230.67 | 256.67 | 250.33 | 263.67 | 246.33 | 293.67 | 344.33 | 373.00 | 430.00 | 432.67 | 476.00 |
| | ST DEV | 142.99 | 117.32 | 130.42 | 148.14 | 120.90 | 115.70 | 150.84 | 155.73 | 180.85 | 195.32 | 234.81 | 230.31 | 232.74 |
| | SEM | 101.11 | 82.96 | 92.22 | 104.75 | 85.49 | 81.81 | 106.66 | 110.11 | 127.88 | 138.11 | 166.04 | 162.86 | 164.57 |
| Group 5 - | 113 | 274 | 198 | 154 | 148 | 157 | 186 | 183 | 162 | 151 | 158 | 171 | 320 | 444 |
| 2.0% | 114 | 147 | 154 | 171 | 316 | 197 | 166 | 152 | 133 | 138 | 190 | 354 | 385 | 449 |
| Alka Vita | 115 | 343 | 346 | 299 | 205 | 337 | 328 | 169 | 139 | 185 | 140 | 398 | 465 | 528 |
| | MEAN | 254.67 | 232.67 | 208.00 | 223.00 | 230.33 | 226.67 | 168.00 | 144.67 | 158.00 | 162.67 | 307.67 | 390.00 | 473.67 |
| | ST DEV | 99.42 | 100.58 | 79.27 | 85.43 | 94.52 | 88.33 | 15.52 | 15.31 | 24.27 | 25.32 | 120.38 | 72.63 | 47.12 |
| | SEM | 70.30 | 71.12 | 56.05 | 60.41 | 66.83 | 62.46 | 10.98 | 10.82 | 17.16 | 17.91 | 85.12 | 51.36 | 33.32 |

TABLE 2

| | |
|---|---|
| Study Number: | 04-290-001 |
| Date of Study: | 11 Aug-21 Sep 2004 |
| Animals Used in Study: | ZDF-obese males |
| Date of Birth: | 17-Jun-04 |

| | | | Rats dosed 21 Sep 04 | | | | | |
|---|---|---|---|---|---|---|---|---|
| | | | at 7:30am | | | Glucose mg/dl | | |
| Rats fasted 20 Sep 04 at 3:30pm | | | Pre Dose (Time 0) | | | Minutes post Dose | | |
| 20-Sep | Animal ID | Body Weight (g) | Chol | Glu | Trig | 30 | 60 | 120 |
| Group 1 - RO | 101 | 381 | 102 | 311 | 1176 | 571 | 627 | 541 |
| H2O | 102 | 352 | 103 | 399 | 697 | 545 | 600 | 541 |
| | 103 | 374 | 97 | 289 | 1169 | 532 | 569 | 457 |
| | MEAN | 369.00 | 100.67 | 333.00 | 1014.00 | 549.33 | 598.67 | 513.00 |
| | ST DEV | 15.13 | 3.21 | 58.21 | 274.55 | 19.86 | 29.02 | 48.50 |
| | SEM | 10.70 | 2.27 | 41.16 | 194.14 | 14.04 | 20.52 | 34.29 |
| Group 2 - 0.5% | 104 | 363 | 96 | 309 | 416 | 569 | 563 | 450 |
| Alka Vita | 105 | 364 | 97 | 453 | 860 | 596 | 623 | 578 |
| | 106 | 344 | 103 | 277 | 711 | 443 | 546 | 439 |
| | MEAN | 357.00 | 98.67 | 346.33 | 662.33 | 536.00 | 577.33 | 489.00 |
| | ST DEV | 11.27 | 3.79 | 93.75 | 225.97 | 81.66 | 40.45 | 77.27 |
| | SEM | 7.97 | 2.68 | 66.29 | 159.78 | 57.75 | 28.60 | 54.64 |
| Group 3 - 1.0% | 107 | 399 | 68 | 160 | 781 | 352 | 408 | 253 |
| Alka Vita | 108 | 332 | 87 | 124 | 432 | 261 | 308 | 208 |
| | 109 | 372 | 63 | 149 | 815 | 383 | 431 | 240 |
| | MEAN | 367.67 | 72.67 | 144.33 | 676.00 | 332.00 | 382.33 | 233.67 |
| | ST DEV | 33.71 | 12.66 | 18.45 | 211.99 | 63.41 | 65.39 | 23.16 |
| | SEM | 23.84 | 8.95 | 13.04 | 149.90 | 44.84 | 46.24 | 16.38 |
| Group 4 - 1.5% | 110 | 391 | 70 | 174 | 1029 | 412 | 415 | 214 |
| Alka Vita | 111 | 353 | 137 | 379 | 886 | 615 | 646 | 540 |
| | 112 | 282 | 107 | 484 | 592 | 693 | 667 | 601 |
| | MEAN | 342.00 | 104.67 | 345.67 | 835.67 | 573.33 | 576.00 | 451.67 |
| | ST DEV | 55.33 | 33.56 | 157.67 | 222.81 | 145.06 | 139.82 | 208.07 |
| | SEM | 39.12 | 23.73 | 111.49 | 157.55 | 102.57 | 98.87 | 147.13 |
| Group 5 - 2.0% | 113 | 388 | 91 | 236 | 1190 | 485 | 541 | 389 |
| Alka Vita | 114 | 378 | 91 | 213 | 833 | 497 | 524 | 405 |
| | 115 | 324 | 95 | 224 | 516 | 604 | 553 | 425 |
| | MEAN | 363.33 | 92.33 | 224.33 | 846.33 | 528.67 | 539.33 | 406.33 |
| | ST DEV | 34.43 | 2.31 | 11.50 | 337.20 | 65.52 | 14.57 | 18.04 |
| | SEM | 24.34 | 1.63 | 8.13 | 238.43 | 46.33 | 10.30 | 12.75 |

TABLE 3

| | |
|---|---|
| Study Number: | 04-290-001 |
| Date of Study: | 11 Aug-21 Sep 2004 |
| Animals Used in Study: | ZDF-obese males |
| Date of Birth: | 17-Jun-04 |

| | | Triglicerides (mg/dl) | | | | | | | | |
|---|---|---|---|---|---|---|---|---|---|---|
| | ANIMAL | 12-Aug | | 16-Aug | 18-Aug | 23-Aug | 30-Aug | 7-Sep | 13-Sep | 20-Sep |
| | ID | 0 TIME | 8 HOUR | DAY 5 | DAY 7 | DAY 12 | DAY 19 | DAY 28 | DAY 34 | DAY 41 |
| Group 1 - RO | 101 | 256 | 316 | 426 | 440 | 680 | 818 | 1267 | 952 | 887 |
| H2O | 102 | 689 | 683 | 609 | 503 | 683 | 908 | 923 | 1006 | 767 |
| | 103 | 607 | 529 | 600 | 484 | 900 | 850 | 1078 | 1397 | 897 |
| | MEAN | 517.33 | 509.33 | 545.00 | 475.67 | 754.33 | 858.67 | 1089.33 | 1118.33 | 850.33 |
| | ST DEV | 230.01 | 184.29 | 103.16 | 32.32 | 126.16 | 45.62 | 172.28 | 242.84 | 72.34 |
| | SEM | 162.64 | 130.31 | 72.94 | 22.85 | 89.21 | 32.26 | 121.82 | 171.71 | 51.15 |
| Group 2 - 0.5% | 104 | 428 | 378 | 388 | 491 | 692 | 1060 | 1011 | 753 | 704 |
| Alka Vita | 105 | 775 | 922 | 656 | 601 | 1147 | 1405 | 1060 | 953 | 844 |
| | 106 | 373 | 448 | 383 | 295 | 599 | 956 | 993 | 997 | 943 |
| | MEAN | 525.33 | 582.67 | 475.67 | 462.33 | 812.67 | 1140.33 | 1021.33 | 901.00 | 830.33 |
| | ST DEV | 217.96 | 295.95 | 156.19 | 155.00 | 293.25 | 235.03 | 34.67 | 130.05 | 120.08 |
| | SEM | 154.12 | 209.27 | 110.45 | 109.60 | 207.36 | 166.19 | 24.52 | 91.96 | 84.91 |
| Group 3 - 1.0% | 107 | 384 | 509 | 383 | 576 | 536 | 854 | 1055 | 678 | 1053 |
| Alka Vita | 108 | 255 | 435 | 261 | 357 | 427 | 474 | 419 | 523 | 615 |
| | 109 | 345 | 347 | 553 | 407 | 638 | 875 | 925 | 1258 | 952 |
| | MEAN | 328.00 | 430.33 | 399.00 | 446.67 | 533.67 | 734.33 | 799.67 | 819.67 | 873.33 |
| | ST DEV | 66.16 | 81.10 | 146.66 | 114.76 | 105.52 | 225.70 | 336.01 | 387.44 | 229.35 |
| | SEM | 46.78 | 57.35 | 103.70 | 81.15 | 74.61 | 159.59 | 237.60 | 273.96 | 162.18 |
| Group 4 - 1.5% | 110 | 530 | 540 | 317 | 294 | 537 | 948 | 955 | 813 | 1316 |
| Alka Vita | 111 | 580 | 646 | 724 | 666 | 930 | 1305 | 1028 | 1042 | 971 |
| | 112 | 552 | 631 | 1045 | 1042 | 1145 | 1107 | 942 | 1285 | 853 |
| | MEAN | 554.00 | 605.67 | 695.33 | 667.33 | 870.67 | 1120.00 | 975.00 | 1046.67 | 1046.67 |
| | ST DEV | 25.06 | 57.36 | 364.85 | 374.00 | 308.31 | 178.85 | 46.36 | 236.03 | 240.60 |
| | SEM | 17.72 | 40.56 | 257.98 | 264.46 | 218.01 | 126.47 | 32.78 | 166.90 | 170.13 |
| Group 5 - 2.0% | 113 | 628 | 538 | 616 | 515 | 549 | 856 | 946 | 1015 | 1211 |
| Alka Vita | 114 | 325 | 316 | 399 | 481 | 762 | 732 | 889 | 1035 | 1264 |
| | 115 | 565 | 664 | 516 | 563 | 607 | 820 | 983 | 1107 | 968 |
| | MEAN | 506.00 | 506.00 | 510.33 | 519.67 | 639.33 | 802.67 | 939.33 | 1052.33 | 1147.67 |
| | ST DEV | 159.88 | 176.19 | 108.61 | 41.20 | 110.12 | 63.79 | 47.35 | 48.39 | 157.84 |
| | SEM | 113.06 | 124.59 | 76.80 | 29.13 | 77.87 | 45.11 | 33.48 | 34.22 | 111.61 |

TABLE 4

| | |
|---|---|
| Study Number: | 04-290-001 |
| Date of Study: | 11 Aug-21 Sep 2004 |
| Animals Used in Study: | ZDF-obese males |
| Date of Birth: | 17-Jun-04 |

| | | Cholesterol (mg/dl) | | | | | | |
|---|---|---|---|---|---|---|---|---|
| | ANIMAL | 12-Aug | | 23-Aug | 30-Aug | 7-Sep | 13-Sep | 20-Sep |
| | ID | 0 TIME | 8 HOUR | DAY 12 | DAY 19 | DAY 28 | DAY 34 | DAY 41 |
| | 101 | 78 | 66 | 68 | 101 | 115 | 123 | 138 |
| Group 1 - RO | 102 | 73 | 67 | 88 | 104 | 107 | 107 | 120 |
| H2O | 103 | 73 | 67 | 86 | 103 | 116 | 111 | 111 |
| | MEAN | 74.67 | 66.67 | 80.67 | 102.67 | 112.67 | 113.67 | 123 |
| | ST DEV | 2.89 | 0.58 | 11.02 | 1.53 | 4.93 | 8.33 | 13.75 |
| | SEM | 2.04 | 0.41 | 7.79 | 1.08 | 3.49 | 5.89 | 9.72 |
| | 104 | 66 | 60 | 81 | 95 | 110 | 88 | 116 |
| Group 2 - 0.5% | 105 | 72 | 73 | 96 | 114 | 117 | 114 | 116 |
| Alka Vita | 106 | 84 | 68 | 89 | 97 | 110 | 107 | 118 |
| | MEAN | 74 | 67 | 88.67 | 102 | 112.33 | 103 | 116.67 |
| | ST DEV | 9.17 | 6.56 | 7.51 | 10.44 | 4.04 | 13.45 | 1.15 |
| | SEM | 6.48 | 4.64 | 5.31 | 7.38 | 2.86 | 9.51 | 0.82 |
| | 107 | 72 | 67 | 83 | 97 | 110 | 97 | 92 |
| Group 3 - 1.0% | 108 | 62 | 61 | 87 | 113 | 107 | 107 | 104 |
| Alka Vita | 109 | 78 | 67 | 90 | 93 | 103 | 99 | 85 |
| | MEAN | 70.67 | 65 | 86.67 | 101 | 106.67 | 101 | 93.67 |
| | ST DEV | 8.08 | 3.46 | 3.51 | 10.58 | 3.51 | 5.29 | 9.61 |
| | SEM | 5.72 | 2.45 | 2.48 | 7.48 | 2.48 | 3.74 | 6.79 |
| | 110 | 78 | 69 | 88 | 91 | 97 | 93 | 93 |
| Group 4 - 1.5% | 111 | 68 | 66 | 85 | 108 | 128 | 142 | 150 |
| Alka Vita | 112 | 64 | 63 | 95 | 118 | 131 | 132 | 127 |
| | MEAN | 70 | 66 | 89.33 | 105.67 | 118.67 | 122.33 | 123.33 |
| | ST DEV | 7.21 | 3 | 5.13 | 13.65 | 18.82 | 25.89 | 28.68 |
| | SEM | 5.1 | 2.12 | 3.63 | 9.65 | 13.31 | 18.31 | 20.28 |
| | 113 | 70 | 60 | 77 | 90 | 99 | 95 | 101 |
| Group 5 - 2.0% | 114 | 63 | 59 | 91 | 87 | 88 | 105 | 116 |
| Alka Vita | 115 | 69 | 68 | 93 | 102 | 118 | 120 | 112 |
| | MEAN | 67.33 | 62.33 | 87 | 93 | 101.67 | 106.67 | 109.67 |
| | ST DEV | 3.79 | 4.93 | 8.72 | 7.94 | 15.18 | 12.58 | 7.77 |
| | SEM | 2.68 | 3.49 | 6.16 | 5.61 | 10.73 | 8.9 | 5.49 |

TABLE 5

| | |
|---|---|
| Study Number: | 04-290-001 |
| Date of Study: | 11 Aug-21 Sep 2004 |
| Animals Used in Study: | ZDF-obese males |
| Date of Birth: | 17-Jun-04 |

| | | Body Weight (g) | | | | | | | |
|---|---|---|---|---|---|---|---|---|---|
| | | 11-Aug | 12-Aug | 18-Aug | 25-Aug | 1-Sep | 8-Sep | 15-Sep | 20-Sep |
| | ANIMAL ID | DAY 0 | DAY 1 | DAY 7 | DAY 14 | DAY 21 | DAY 28 | DAY 35 | DAY 40 |
| Group 1 - RO | 101 | 253 | 258 | 292 | 341 | 361 | 380 | 406 | 417 |
| H2O | 102 | 256 | 266 | 293 | 331 | 347 | 371 | 373 | 378 |
| | 103 | 261 | 265 | 290 | 333 | 350 | 374 | 394 | 397 |
| | MEAN | 256.67 | 263.00 | 291.67 | 335.00 | 352.67 | 375.00 | 391.00 | 397.33 |
| | ST DEV | 4.04 | 4.36 | 1.53 | 5.29 | 7.37 | 4.58 | 16.70 | 19.50 |
| | SEM | 2.86 | 3.08 | 1.08 | 3.74 | 5.21 | 3.24 | 11.81 | 13.79 |
| Group 2 - 0.5% | 104 | 268 | 272 | 302 | 351 | 369 | 383 | 384 | 393 |
| Alka Vita | 105 | 279 | 282 | 306 | 344 | 358 | 373 | 384 | 385 |
| | 106 | 236 | 242 | 269 | 311 | 325 | 349 | 367 | 376 |
| | MEAN | 261.00 | 265.33 | 292.33 | 335.33 | 350.67 | 368.33 | 378.33 | 384.67 |
| | ST DEV | 22.34 | 20.82 | 20.31 | 21.36 | 22.90 | 17.47 | 9.81 | 8.50 |
| | SEM | 15.80 | 14.72 | 14.36 | 15.11 | 16.19 | 12.36 | 6.94 | 6.01 |
| Group 3 - 1.0% | 107 | 259 | 264 | 296 | 333 | 353 | 381 | 405 | 415 |
| Alka Vita | 108 | 278 | 256 | 256 | 278 | 287 | 308 | 324 | 341 |
| | 109 | 242 | 244 | 270 | 310 | 333 | 352 | 378 | 387 |
| | MEAN | 259.67 | 254.67 | 274.00 | 307.00 | 324.33 | 347.00 | 369.00 | 381.00 |
| | ST DEV | 18.01 | 10.07 | 20.30 | 27.62 | 33.84 | 36.76 | 41.24 | 37.36 |
| | SEM | 12.73 | 7.12 | 14.35 | 19.53 | 23.93 | 25.99 | 29.16 | 26.42 |
| Group 4 - 1.5% | 110 | 253 | 257 | 284 | 323 | 340 | 371 | 395 | 410 |
| Alka Vita | 111 | 285 | 290 | 314 | 346 | 360 | 370 | 378 | 391 |
| | 112 | 238 | 248 | 266 | 284 | 295 | 298 | 299 | 306 |
| | MEAN | 258.67 | 265.00 | 288.00 | 317.67 | 331.67 | 346.33 | 357.33 | 369.00 |
| | ST DEV | 24.01 | 22.11 | 24.25 | 31.34 | 33.29 | 41.86 | 51.23 | 55.38 |
| | SEM | 16.98 | 15.64 | 17.15 | 22.16 | 23.54 | 29.60 | 36.22 | 39.16 |
| Group 5 - 2.0% | 113 | 260 | 267 | 288 | 326 | 347 | 379 | 403 | 411 |
| Alka Vita | 114 | 256 | 259 | 290 | 324 | 354 | 382 | 396 | 400 |
| | 115 | 246 | 244 | 261 | 296 | 320 | 335 | 346 | 349 |
| | MEAN | 254.00 | 256.67 | 279.67 | 315.33 | 340.33 | 365.33 | 381.67 | 386.67 |
| | ST DEV | 7.21 | 11.68 | 16.20 | 16.77 | 17.95 | 26.31 | 31.09 | 33.08 |
| | SEM | 5.10 | 8.26 | 11.45 | 11.86 | 12.70 | 18.61 | 21.98 | 23.39 |

For a period between Jan. 3, 2005 and Feb. 18, 2005, a study was conducted to evaluate the antitumor activity of Alka vita on tumor growth in LOX-GFP human melanoma model through intradermal injection of LOX cells in nude mice.

Materials and Methods

The pLEIN vector was purchased from Clontech (Palo Alto, Calif.). The vector expresses enhanced GFP and the neomycin resistance gene on the same bicistronic message that contains an internal ribosome entry site.

PT67, an NIH3T3-derived packaging cell line, expressing the 10 A1 viral envelope, was purchased from Clontech. PT67 cells were cultured in DMEM supplemented with 10% fetal bovine serum. For vector production, packaging cells (PT67), at 70% confluence, were incubated with a precipitated mixture of N-[1-(2,3-dioleoyloxy)propyl]-N,N,N-trimethylammoniummethyl sulfate reagent and saturating amounts of pLEIN plasmid for 18 h. Fresh medium was replenished at this time. The cells were examined by fluorecence microscopy 48 h post-transfection. For selection, the cells were cultured in the presence of 200-1000 μg/ml G418 for 7 days.

For GFP gene transduction, LOX cells (National Cancer Institute, Bethesda, Md.) at 25% confluence were incubated with a 1:1 precipitated mixture of retroviral supernatants of PT67 cells and RPMI 1640 (Life Technologies, Inc.) containing 10% fetal bovine serum (Gemini Bioproducts) for 72 h. Fresh medium was replenished at this time. Cells were harvested by trypsin-EDTA 72 h after transduction and sub-cultured at a ratio of 1:15 into selective medium that contained 200 μg/kg G418. The level of G418 was increased stepwise to 800 μg/kg for LOX cells. Clones expressing GFP were isolated with cloning cylinders (Bel-Art Products, Pequannock, N.J.) using trypsin-EDTA and then amplified and transferred by conventional culture methods.

GFP or non-transduced cells were seeded at 1.5×10⁴ in 35-mm culture dishes. The cells were harvested and counted every 24 h using a hemocytometer (Reichert Scientific Instruments, Buffalo, N.Y.). The doubling time was calculated from the cell growth curve over 6 days.

Fifteen 6-week-old male NCr mice were injected intradermally with a single dose of 1×10⁶ LOX-GFP cells. Cells were first harvested by trypsinization and washed three times with cold serum-free medium and then injected in a total volume of 0.1 ml within 30 min of harvesting. Cells were inoculated into dorsal skin using a 30 G1/2 precision glide needle (Becton Dickinson) and a 1-ml latex-free syringe (Becton Dickinson).

A Leica stereo fluorescence microscope model LZ2 equipped with a mercury lamp power supply was used. Selective excitation of GFP was produced through a D425/60 band-pass filter and 470 DCXR dichroic mirror. Emitted fluorescence was collected through a long-pass filter GG475 (Chroma Technology, Brattleboro, Vt.) on a Hamamatsu C5810 3-chip cooled color CCD camera (Hamamatsu Photonics Systems, Bridgewater, N.J.). Images were processed for contrast and brightness and analyzed with the help of Image Pro Plus 3.1 software (Media Cybernetics, Silver Spring, Md.). High-resolution images were captured directly on the computer or continuously through video output on a high-resolution Sony VCR.

Metastases were detected by direct GFP open imaging at necropsy. Locations of metastases were recorded for each test animal.

The intradermal (orthotopically) injected animals used for the study were divided into 3 groups 4 days after surgery. Groups for each of the cohort conditions were randomly chosed. Treatment began 4 days after implantation three times a day and lasted for six weeks (The concentration of Alkahydroxy B was increased to 25% on January 25^{th}, 2005, lasting 10 days; and the concentration of Alkahydroxy B was increased again to 50% on Feb. 4, 2005, lasting two weeks). Table 6 shows the study design and compounds used in each group.

TABLE 6
Treatment protocol

| | | Route of | Number |
|---|---|---|---|
| Group & Agent | Schedule | administration | of Mice |
| 1, Untreated control | — | — | 5 |
| 2, Alkahydroxy A | Three times a day for 6 | Topical | 5 |
| | weeks | | |
| 3, Alkahydroxy B | Three times a day for 6 | Topical | 5 |
| | weeks | | |

The experiment was terminated 47 days after tumor cell injection (6 weeks treatment) due to the poor health status of the test animals.

Data Collection:

GFP whole body images for each mouse were obtained once a week after initial treatment.
Tumor measurements were determined by GFP whole body imaging.
Body weight for each animal was measured once a week after initial treatment. An electronic balance was used for body weight measurement.
The final primary tumor weight for each animal were determined with an electronic balance.
At the end of the study, GFP open imaging was conducted. The primary tumor and major metastatic organs were explored under fluorescence microscopy at necropsy.

Final primary tumor weights in each group were analyzed using the ANOVA test (with the Dunnett's two sided test) with an α=0.05. Tumor metastatic rates of all groups were analyzed with Fisher's exact test with an α=0.05.

Results

At the end of the study, the final primary tumor weights in treated groups were compared to that of the untreated control group using the Dunnett's two-sided test. Statistical significant differences were obtained in Alkahydroxy A treated group (p<0.05), verses the untreated control group. The results are shown in Table 7.

TABLE 7
Efficacy of Alkahydroxy A & B on final primary tumor
weight

| | Mean final tumor | |
|---|---|---|
| Group | weight (g) | P-value* |
| 1, Untreated control | 8.1 | — |
| 2, Alkahydroxy A | 3.9 | 0.034 |
| 3, Alkyhydroxy B | 4.7 | 0.091 |

*All treated groups are versus the untreated control group with Dunnett's two-sided test.

At the end of the study, all animals were opened and imaged to examine metastasis. Metastatic incidences in different organs were determined by direct GFP open imaging. The metastases and p-value for each organ in treated groups were compared to that of the untreated control group by using Fisher's exact test (Table 8). No statistically significant difference was seen in either treatment compared to the untreated control. The results are shown in Table 8.

TABLE 8
Efficacy on Metastatic rate

| | |
|---|---|
| # of tested | Superficial axillary L.N. |

| Group | animals | MI^{a} | P^{b} |
|---|---|---|---|
| 1 Untreated control | 5 | 0 | — |
| 2 Alkahydroxy A | 5 | 1 | 1.000 |
| 3 Alkyhydroxy B | 5 | 0 | 1.000 |

^{a}MI Metastatic Incidence.
^{b}All treated groups compared to the untreated control group by Fisher's exact test.

No acute loss of body weight within the duration of the experiment was observed (see “Body weight graph”).

### CONCLUSION

The results showed that Alkahydroxy A significantly inhibited the growth of primary tumor (p<0.05), verses the untreated control group. No significant difference was seen regarding metastases for both treated groups compared with the untreated control group. There was no evidence of toxicity as shown in the body weight graph.

Dosage

As noted above, dosages of the present invention may be tailored to meet the specific needs of individual consumers. The method of administration may comprise any convenient route, including but not limited to parenteral, subcutaneous, intravenous, intramuscular, intraperitoneal, or transdermal. Alternatively or concomitantly, administration may be oral. The dosage administered varies with the age, health, weight, and disease condition of the individual patient/consumer. Dosage may also be tailored to accord with any concurrent treatment, if any, and the nature of the effect desired.

Compositions within the scope of the present invention include all compositions wherein the active ingredients are contained in an amount effective to achieve the intended purpose. Although individual needs vary, determination of optimal ranges of effective amounts of each individual component is within the skill of the art. Typical dosages comprise 0.01-100 mg/kg body weight. Preferred dosages comprise 0.1-100 mg/kg body weight. Most preferred dosages comprise 1-50 mg/kg body weight.

Pharmaceutical compositions for administering the active ingredients of the present invention preferably contain, in addition to the pharmacologically active compound, suitable pharmaceutically acceptable carriers. These carriers may comprise any excipients and auxiliaries that facilitate processing and/or preserving the active compounds in pharmaceutical preparations.

Preferably, these preparations are administered orally in the form of aqueous solutions. In still another embodiment, suitable solutions of the present invention may be administered by injection or orally. Any of these forms preferably contain from about 0.01-99% by weight active compounds, together with one or more appropriate excipients. For purposes of the present invention, all percentages are by weight unless otherwise indicated.

Suitable formulations for parenteral administration include aqueous solutions of the active compounds in water-soluble form, such as water-soluble salts.

Any number of assays well known in the art may be used to test whether a particular formulation is effective for the present invention.

In determining dosages of the composition of the present invention to be administered, the dosage and frequency of administration is selected in relation to the pharmacological properties of the specific active ingredients. Normally, at least three dosage levels may be used. In toxicity studies in general, the highest dose should reach a toxic level but be sub-lethal for most animals in the group. If possible, the lowest dose can induce a biologically demonstrable effect. These studies are preferably performed in parallel for each formulation selected.

Additionally, the ID₅₀ level of the composition can be one of the dosage levels selected, and the other two selected to reach a toxic level. The lowest dose would be one which does not exhibit a biologically demonstrable effect. The toxicology tests should be repeated using appropriate new doses calculated on the basis of the results obtained. Young, healthy mice or rats belonging to a well-defined strain are the first choice of species.

When a suitable and presumably safe dosage of the composition has been established, studies on the composition's chronic toxicity, its effect on reproduction, and potential mutagenicity may also be required in order to ensure that the calculated appropriate dosage range will be safe with regard to these hazards.

The amount of the composition according to the present invention to be administered to any given patient must be determined empirically, and will differ depending upon the condition of the patient. Relatively small amounts of the composition can be administered at first, with steadily increasing dosages if no adverse effects are noted. In general, 17% to 2% aqueous solutions of silica are preferred, given once to four times daily.

It is to be understood that the phraseology or terminology employed herein is for the purpose of description and not of limitation. The means and materials for carrying out disclosed functions may take a variety of alternative forms without departing from the invention. Thus, the expressions “means to . . . ” and “means for . . . ” as may be found the specification above, and/or in the claims below, followed by a functional statement, are intended to define and cover whatever structural, physical, chemical, or electrical element or structures which may now or in the future exist for carrying out the recited function, whether or not precisely equivalent to the embodiment or embodiments disclosed in the specification above, and it is intended that such expressions be given their broadest interpretation.

1. A composition for increasing the alkalinity of the body comprising about 1 to about 8 parts by molecular weight of A, about 8 to about 15 parts by molecular weight of B, and about 1 to about 5 parts by molecular weight of C, wherein:
a. A is silicon and an optional metal ion selected from the group consisting of aluminum, tin, zinc, and combinations thereof.
b. B is R.O. water; and
c. C is a source of alkalinity selected from alkaline water and an alkaline hydroxide selected from the group consisting of calcium, lithium, magnesium, potassium, sodium, and mixtures thereof.

2. The composition of matter according to claim 1 wherein the source of alkalinity is alkaline water.

3. A method for administering the compound of claim 1 wherein the method of administration is selected from the group consisting of parenteral, oral, aerosol, parenteral, subcutaneous, intravenous, and combinations thereof.

4. The method according to claim 3 wherein the composition is administered in amounts of 0.1-100 mg/kg body weight.

5. The method according to claim 3 wherein the composition is administered in amounts of 1-50 mg/kg body weight.

6. The method according to claim 3 wherein the composition is administered in amounts sufficient to bring the body's pH into balance.

7. The method according to claim 6 wherein the composition is administered in amounts sufficient to bring the body's internal environment to a pH of 7 to 7.4.

8. A method for treating diseases arising from excess acidity in the body comprising administering to an animal in need thereof an effective amount of the composition according to claim 1.

9. The method according to claim 6 wherein the acidity-related diseases are selected from the group consisting of HIV, cancer, hepatitis C, viruses, diabetes, acid reflux, stomach ailments, yeast infections, genital herpes, STDs, and SARS.

10. A method for reducing or eliminating vitamin or mineral or nutrient deficiencies by administering to an animal in need thereof sufficient amounts of the composition of claim 1 to return the body pH to optimal levels.

11. A method for treating osteoporosis comprising administering to an animal in need thereof an effective amount of the composition of claim 1.

12. The method according to claim 9 wherein the disease is diabetes.

13. The method according to claim 9 wherein the disease is cancer.
